SEQUENCE LISTING (1 sequence)

SEQ ID NO: 1 - DNA, 1900 bp, Artificial Sequence
  Description of Artificial Sequence Note= synthetic construct
ggggcaaataacaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaaggaaaataagtgtaatggaacagatgctaaggtaaaattgataaaacaagaattagataaatataaaaatgctgtaacagaattgcagttgctcatgcaaagcacaccaccaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaaataaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaagggatggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaaataaaaatagcacctaatcatgttcttacaatggtttactatctgctcatagacaacccatctgtcattggattttcttaaaatctgaacttcatcgaaactctcatctataaaccatctcacttacactatttaagtagattcctagtttatagttatata

(19) United States
(12) Patent Application Publication
(10) Pub. No.: US 2004/0009903 A1
(43) Pub. Date: Jan. 15, 2004

(54) Macroaggregated albumin-polyethyleneimine (MAA-PEI) lung-targeted delivery of respiratory syncytial virus DNA vaccines

(76) Inventors: Ralph A. Tripp, Decatur, GA (US); Jennifer L. Harcourt, Lilburn, GA (US)

Correspondence Address: NEEDLE & ROSENBERG, P.C., SUITE 1000, 999 PEACHTREE STREET, ATLANTA, GA, 30309-3915, US

(21) Appl. No.: 10/453,219

(22) Filed: Jun. 2, 2003

Related U.S. Application Data
(60) Provisional application No. 60/384,586, filed on May 31, 2002.

(51) Int. Cl. A61K 48/00

(52) U.S. Cl. 514/8; 514/44

(57) ABSTRACT

The present invention provides a composition comprising: 1) macroaggregated albumin, 2) a nucleic acid comprising a nucleotide sequence encoding an RSV protein, and 3) polyethylamine (PEI), wherein the MAA, PEI and nucleic acid form a complex. Also provided by the present invention is a method of preventing respiratory syncytial virus (RSV) infection in a subject comprising administering to the subject an amount of a composition of this invention.

This application claims the benefit of priority of U.S. Provisional Application No. 60/384,586, filed May 31, 2002. The 60/384,586 provisional patent application is herein incorporated by this reference in its entirety.

### FIELD OF THE INVENTION

This invention relates to compositions and methods for preventing respiratory syncytial virus (RSV) infection.

### BACKGROUND OF THE INVENTION

Currently, no safe and effective RSV vaccine is available. DNA vaccines encoding RSV F or G glycoproteins are one RSV vaccine option being examined for safety and efficacy. However, DNA vaccination has been problematic because of the need for repeated vaccination requiring large amounts of DNA, and DNA vaccination does not often result in mucosal immunity.

In order to overcome these limitations, the present invention provides, for the first time, compositions comprising: 1) macroaggregated albumin, 2) a nucleic acid comprising a nucleotide sequence encoding an RSV glycoprotein and 3) polyethylamine (PEI), wherein the MAA, PEI and nucleic acid form a complex as well as methods for delivering these compositions to a subject to prevent RSV infection.

### SUMMARY OF THE INVENTION

The present invention provides a composition comprising: 1) macroaggregated albumin, 2) a nucleic acid comprising a nucleotide sequence encoding an RSV protein, and 3) polyethylamine (PEI), wherein the MAA, PEI and nucleic acid form a complex.

Also provided by the present invention is a method of preventing respiratory syncytial virus (RSV) infection in a subject, comprising administering to the subject a composition comprising: 1) macroaggregated albumin, 2) a nucleic acid comprising a nucleotide encoding an RSV protein, and 3) polyethylamine (PEI), wherein the MAA, PEI and nucleic acid form a complex, in an amount effective in preventing RSV infection in the subject.

Further provided is a method of producing and/or enhancing an immune response to respiratory syncytial virus (RSV) in a subject, comprising administering to the subject a composition comprising: 1) macroaggregated albumin, 2) a nucleic acid comprising a nucleotide sequence encoding an RSV protein, and 3) polyethylamine (PEI), wherein the MAA, PEI and nucleic acid form a complex, in an amount effective in producing and/or enhancing the immune response to RSV in the subject.

### BRIEF DESCRIPTION OF THE DRAWINGS

FIG. 1 shows that MAA-F vaccination induces an anti-F glycoprotein serum antibody response. An ELISA was used to determine anti-RSV F glycoprotein antibody titers in sera diluted 1:100 from six individual mice. Serum was collected at the day of challenge (pre), or day 12 or 14 post-challenge from 2X MAA or 2X MAA-F vaccinated mice (A), or from 1X, 10 μg MAA or MAA-F vaccinated mice (B). Anti-RSV-F glycoprotein antibody responses reactive to purified RSV-F glycoprotein [64] are expressed as the average absorbance value ± standard deviation.

FIG. 2 shows that MAA-F vaccination induces a neutralizing antibody response. Serum neutralization studies were performed to determine the levels of neutralizing antibodies associated with 1X MAA or MAA-F vaccination (B), 2X MAA or MAA-F vaccination (C), or 1X, 10 μg MAA or MAA-F vaccination (D) in sera collected at day 14 post-RSV/A2 challenge. RSV hyperimmune serum (A) was used as a positive control. The data is presented as the mean percent of inhibition of RSV plaque formation ± standard deviation, from 3 replicates of 5-6 serum samples per vaccine group. Statistically different values (p<0.05) between MAA and MAA-F vaccinated mice were determined by unpaired two-tailed analysis, and are indicated by an asterisk (*).

FIG. 3 shows that MAA-F vaccination is associated with intracellular Th1-type cytokine expression. The total number of CD3⁺ BAL cells expressing Thl-type cytokines was determined for 1X MAA or MAA-F vaccinated mice (A, D, G), 2X MAA or MAA-F vaccinated mice (B, E, H), or 1X, 10 μg MAA or MAA-F vaccinated mice (C, F, I) following RSV/A2 challenge. The total number of CD3⁺ IL-2⁺ (A, B, C), CD3⁺ IFNγ⁺ (D, E, F) and CD3⁺ TNFα⁺ (G, H, I) BAL cells was determined by flow cytometry from 3 separate BAL samples pooled from 2 mice/treatment (6 mice for each time point). Data represents average total number of cells ± standard deviation. Statistically different values (p<0.05) between MAA and MAA-F vaccinated mice were determined by unpaired two-tailed analysis, and are indicated by an asterisk (*).

FIG. 4 shows that MAA-F vaccination is associated with intracellular Th2-type cytokine expression. The total number of CD3⁺ BAL cells expressing Th2-type cytokines was determined for 1X MAA or MAA-F vaccinated mice (A, D, G), 2X MAA or MAA-F vaccinated mice (B, E, H), or 1X, 10 μg MAA or MAA-F vaccinated mice (C, F, I) following RSV/A2 challenge. The total number of CD3⁺ IL-4⁺ (A, B, C), CD3⁺ IL-5⁺ (D, E, F) and CD3⁺ IL-10⁺ (G, H, I) BAL cells was determined by flow cytometry from 3 separate BAL samples pooled from 2 mice/treatment (6 mice for each time point). Data represents average total number of cells ± standard deviation. Statistically different values (p<0.05) between MAA and MAA-F vaccinated mice were determined by unpaired two-tailed analysis, and are indicated by an asterisk (*).

FIG. 5 shows that MAA-F vaccination is not associated with significant pulmonary cellular inflammation. The total number of CD4⁺ T cells (A, B, C), CD8⁺ T cells (D, E, F) and B220⁺ cells (G, H, I) were determined in BAL cells from 1X MAA or MAA-F vaccinated mice (A, D, G), 2X MAA or MAA-F vaccinated mice (B, E, H), or 1X, 10 μg MAA or MAA vaccinated mice (C, F, I) challenged with RSV/A2. Data represents the average total number of cells ± standard deviation for 6 mice/time-point. Statistically different values (p<0.05) between MAA and MAA-F vaccinated mice were determined by unpaired two-tailed analysis, and are indicated by an asterisk (*).

FIG. 6 shows that MAA-F vaccination is not associated with significant pulmonary granular cell inflammation. The total number of DX5⁺ NK cells (A, B, C), RB6-8C5⁺ neutrophils (D, E, F) and CD11b⁺ cells (G, H, I) were determined in BAL cells from 1X MAA or MAA-F vaccinated mice (A, D, G), 2X MAA or MAA-F vaccinated mice (B, E, H), or 1X, 10 μg MAA or MAA-F vaccinated mice challenged with RSV/A2. Data represents the average total number of cells ± standard deviation for 6 mice/time-point. Statistically different values (p<0.05) between MAA and MAA-F vaccinated mice were determined by unpaired two-tailed analysis, and are indicated by an asterisk (*).

### DETAILED DESCRIPTION OF THE INVENTION

The present invention may be understood more readily by reference to the following detailed description of the preferred embodiments of the invention and the Example included herein.

Before the present methods are disclosed and described, it is to be understood that this invention is not limited to specific nucleic acids or specific methods. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise.

The present invention provides a composition comprising 1) macroaggregated albumin, 2) a nucleic acid comprising a nucleotide sequence encoding an RSV protein and 3) polyethylamine (PEI), wherein the MAA, PEI and nucleic acid form a complex.

The complexes of the present invention can be made as described in the Examples included herein. For example, MAA-PEI particles can be prepared as described in Orson et al. (Journal of Immunology, 164: 6313-21, 2000). The Orson et al. reference is incorporated herein in its entirety, by this reference, for the purposes of describing how MAA-PEI particles can be prepared. Once MAA-PEI particles are prepared, the MAA-PEI particles can be combined with a nucleic acid to obtain a MAA-PEI-nucleic acid complex. Alternatively, the particles can be made by 1) making MAA particles by methods standard in the art; 2) making a PEI-nucleic acid complex, and 3) combining the MAA particles and the PEI-nucleic acid complex to obtain the MAA-PEI-nucleic acid particles of the present invention. Also, MAA particles, PEI particles and nucleic acids can be combined to obtain the MAA-PEI-nucleic acid particles of the present invention.

As used herein, the term “nucleic acid” refers to single-or multiple stranded molecules that may be DNA or RNA, or any combination thereof, including modifications to those nucleic acids. The nucleic acid may represent a coding strand or its complement, or any combination thereof. Nucleic acids may be identical in sequence to the sequences which are naturally occurring for any of the RSV proteins discussed herein or may include alternative codons which encode the same amino acid as that which is found in the naturally occurring sequence. These nucleic acids can also be modified from their typical structure. Such modifications include, but are not limited to, methylated nucleic acids, the substitution of a non-bridging oxygen on the phosphate residue with either a sulfur (yielding phosphorothioate deoxynucleotides), selenium (yielding phosphorselenoate deoxynucleotides), or methyl groups (yielding methylphosphonate deoxynucleotides).

The nucleic acids of the present invention comprise a nucleic acid encoding an RSV protein or a portion or fragment or domain of an RSV protein. Examples of RSV proteins include, but are not limited to RSV glycoproteins, (such as an F glycoprotein or a G glycoprotein), M protein, N protein, 1B protein and 1C protein.

In order to express an RSV protein or portion or fragment or domain thereof, the nucleic acid can include, for example, expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from metallothionine genes, actin genes, immunoglobulin genes, CMV, SV40, adenovirus, bovine papilloma virus, etc. The nucleic acids can be generated by means standard in the art, such as by recombinant nucleic acid techniques and by synthetic nucleic acid synthesis or in vitro enzymatic synthesis.

The nucleic acid of this invention can be in the form of naked DNA or RNA. The nucleic acid can also be a plasmid or a vector comprising a nucleic acid encoding an RSV protein. The nucleic acid can also be a viral vector comprising a nucleic acid encoding an RSV protein of this invention. One skilled in the art will appreciate that the viral vector utilized can comprise any viral vector amenable to delivery to lung cells and production of the RSV protein or portion, fragment or domain thereof. For example, the viral vector can be a recombinant adenovirus vector (71), an adeno-associated viral vector (72), a lentiviral vector (73) a pseudotyped retroviral vector (74), a vaccinia vector, an alphavirus vector, or any other viral vector known in the art.

As noted above, the viral vector of this invention can be a retrovirus. The retrovirus of this invention can be in the Oncovirinae subfamily of retroviruses, such as HTLV-I or HTLV-II (human T-cell leukemia virus type I and type II, respectively). Additionally, the retrovirus can be in the Lentivirinae subfamily of retroviruses, such as HIV-1, HIV-II, SIV, FIV, EIAV and CAEV (human immunodeficiency virus type I, human immunodeficiency virus type II, simian immunodeficiency virus, feline immunodeficiency virus, equine infectious anemia virus, and caprine arthritis-encephalitis virus, respectfully).

In an embodiment where the viral vector is an adenovirus, the nucleic acid can comprise an entire wild-type adenoviral genome or a mutant thereof, or a construct wherein the only adenoviral sequences present are those which enable the nucleic acid to be packaged into an adenovirus particle, or any variation thereof. Packageable lengths of nucleic acids are known in the art. This adenoviral genome can be coupled with any desired nucleic acid encoding an RSV protein of this invention such that the adenoviral genome, when packaged into an adenovirus particle, also packages the nucleic acid insert. One skilled in the art will appreciate that the nucleic acid insert combined with the adenoviral nucleic acid will be of a total nucleic acid length that will allow the total nucleic acid to be packaged into an adenovirus particle.

The nucleic acids encoding an RSV protein can be from A or B strains of RSV. The nucleic acids can be full-length coding sequences for an RSV protein or the nucleic acids can encode a fragment or portion or domain of an RSV protein. For example, based on the teachings of the present invention, one of skill in the art would be able to obtain a fragment of an RSV glycoprotein and determine whether or not a fragment is effective in preventing an RSV infection and/or effective in producing and/or enhancing an immune response to RSV. The nucleic acids encoding an RSV glycoprotein of the present invention include, but are not limited to, an RSV/A Long F glycoprotein, an RSV/A Long G glycoprotein, an RSV/6340 G glycoprotein, an RSV/6340 F glycoprotein, an RSV/A2 F glycoprotein, and RSV/A2-G glycoprotein, an RSV/A-WV6973 G glycoprotein, an RSV/A-WV6973 F glycoprotein, an RSV/B1 G glycoprotein, an RSV/B1 F glycoprotein, an RSV/B-8/60 G glycoprotein, an RSV/B-8/60 F glycoprotein, an RSV/B-18537 G glycoprotein, an RSV/B-18537 F glycoprotein, a cpts-248 RSV G glycoprotein, a cpts-248 RSV F glycoprotein, a cpts-530 RSV G glycoprotein, a cpts-530 RSV F glycoprotein, a Ts-1 RSV G glycoprotein, a Ts-1 RSV F glycoprotein, a bovine RSV/A1 G glycoprotein, and a bovine RSV/A1 F glycoprotein

Nucleic acids encoding an RSV glycoprotein or a fragment, portion or domain thereof, can be administered in combination with other nucleic acids encoding RSV proteins or fragments, portions or domains thereof, such as nonstructural protein 1C, nonstructural protein 1B, major nucleocapsid (N), phosphoprotein (P), protein M, 1A (1A) and envelope associated protein (22K). The nucleic acid sequences encoding these RSV/A2 proteins are available via GENBANK ACCESSION NOs. M11486 K01459 K02719 K03348 K03349 M11217 M11244 M11487 M11505 M11514 M11631 M12966. These accession numbers provide the complete coding sequences for human respiratory syncytial virus nonstructural protein (1C), nonstructural protein (1B), major nucleocapsid (N), phosphoprotein (P), protein (M), 1A (1A), protein G (G), protein (F) and envelope-associated protein (22K) gene which sequences are incorporated in their entireties by this reference. The nucleotide sequence of the gene encoding the fusion (F) glycoprotein of human respiratory synctial virus strain A2 can also be found in Collins et al. “Nucleotide sequence of the gene encoding the fusion (F) glycoprotein of human respiratory syncytial virus,” Proc. Natl. Acad. Sci USA 81: 7683-7687 (1984). This reference and the sequence of RSV A2 F glycoprotein included therein are incorporated in their entireties by this reference.

A nucleic acid sequence encoding bovine RSV/A1 F glycoprotein is available via GENBANK ACCESSION NO: AF188555 and this sequence is incorporated in its entirety by this reference; a nucleic acid sequence encoding bovine RSV/A1 G glycoprotein is available via GENBANK ACCESSION NO: AF188588 and this sequence is incorporated in its entirety by this reference; a nucleic acid sequence encoding human RSV/A long G protein is available via GENBANK ACCESSION NO: M17212 and this sequence is incorporated in its entirety by this reference; a nucleic acid sequence encoding the complete genome of human RSV/B1 is available via GENBANK ACCESSION NO: AF013254 and this sequence is incorporated in its entirety by this reference; a nucleic acid sequence encoding the human RSV/B-8/60 G protein is available via GENBANK ACCESSION NO: M55633 and this sequence is incorporated in its entirety by this reference.

The present invention also provides a method of treating or preventing respiratory syncytial virus (RSV) infection in a subject comprising administering to the subject a composition comprising: 1) macroaggregated albumin, 2) a nucleic acid comprising a nucleotide sequence encoding an RSV protein, and 3) polyethylamine (PEI), wherein the MAA, PEI and nucleic acid form a complex, in an amount effective in treating or preventing RSV infection in the subject.

By “treating” is meant that an improvement in the disease state, i.e., RSV infection, is observed and/or detected upon administration of a composition of the present invention to a subject. Treatment can range from a positive change in a symptom or symptoms of the disease to complete amelioration of RSV infection (e.g., reduction in severity or intensity of disease, alteration of clinical parameters indicative of the subject's condition, relief of discomfort or increased or enhanced function), as detected by art-known techniques.

By “preventing” is meant that after administration of a composition of the present invention to a subject, the subject acquires an immune response which can be detected by art known methods and which is protective, and wherein subsequent challenge with RSV does not result in a clinically manifested RSV infection.

Also provided by the present invention is a method of producing and/or enhancing an immune response to respiratory syncytial virus (RSV) in a subject, comprising administering to the subject a composition comprising: 1) macroaggregated albumin, 2) a nucleic acid comprising a nucleotide sequence encoding an RSV protein, and 3) polyethylamine (PEI), wherein the MAA, PEI and nucleic acid form a complex, in an amount effective in producing and/or enhancing an immune response to RSV in the subject.

As used herein, “an immune response to RSV” includes Th1 and/or Th2 cytokine responses as well as an increase in anti-RSV antibodies in the subject. For example, one of skill in the art would be able to measure the amount of anti-RSV antibodies in the serum of a subject and determine the extent of an immune response to RSV.

The methods of the present invention can be utilized to deliver nucleic acids encoding RSV glycoproteins and/or portions, fragments or domains thereof, and/or other RSV proteins and/or portions, fragments or domains thereof to the lung in a subject. In one embodiment, one skilled in the art could utilize the methods of the present invention to administer the compositions of this invention to the lung of a subject in vivo and/or ex vivo according to standard methods.

If ex vivo methods are employed, cells or tissues can be removed and maintained outside the body according to standard protocols well known in the art. The compositions of this invention can be introduced into the cells via any gene transfer mechanism, such as, for example, virus-mediated gene delivery, calcium phosphate mediated gene delivery, electroporation, microinjection or proteoliposomes. The cells containing the nucleic acid can then be infused (e.g., in a pharmaceutically acceptable carrier) or transplanted back into a subject per standard methods for the cell or tissue type. Standard methods are known for transplantation or infusion of various cells into a subject.

For either ex vivo or in vivo use, the compositions of this invention can be. administered at any effective concentration. An effective concentration or amount of a composition is one that results in prevention of an RSV infection and/or in an immune response to RSV. One skilled in the art would know how to determine an effective concentration or amount according to methods known in the art, as well as provided herein. For example, for targeting to the lung, lung cells can be utilized to determine optimal dosages for delivery of the nucleic acids of this invention. By conducting these experiments in vitro, one of skill in the art can optimize the in vivo dosage of a particular nucleic acid, including concentration and time course of administration.

One of skill in the art would know how to determine the amount of RSV nucleic acid or fragment thereof that is to be administered based on methods known in the art and the teachings of the present invention. See Comerota et al. “Naked plasmid DNA encoding fibroblast growth factor type I for the treatment of end-stage unreconstructible lower extremity eschemia: preliminary results of a phase I trial” J. Vasc. Surg. 35: 930-936 (2002)). As an example, one of skill in the art could utilize 0.5 μg, 1 μg, 2 μg, 3 μg, 4 μg, 5 μg, 6 μg, 7 μg, 8 μg, 9 μg, 10 μg, 20 μg, 30 μg, 40 μg, 50 μg, 60 μg, 70 μg, 80 μg, 90 Ξg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 750 μg, 1,000 μg, 1,250 μg, 1,500 μg, 1,750 μg, 2,000 μg, 3,000 μg, 4,000 μg, 5,000 μg, 6,000 μg, 7,000 μg, 8,000 μg, 9,000 μg, 10,000 μg, 11,000 μg, 12,000 μg, 13,000 μg, 14,000 μg, 15,000 μg, 16,000 μg, 17,000 μg, 18,000 μg, 19,000 μg, 20,000 μg, 30,000 μg, 50,000 μg or any other amount of an RSV nucleic acid between 0.5 μg and 50,000 μg to make any of the compositions of the present invention. Such nucleic acids can be administered as a single dosage or in repeated dosages.

Dosages of the compositions of the present invention will also depend upon the type and/or severity of RSV infection and the individual subject's status (e.g., species, weight, disease state, etc.) Dosages will also depend upon the form of the composition being administered and the mode of administration. Such dosages are known in the art or can be determined as described above. For example, if the nucleic acid of this invention is delivered to the cells of a subject in an adenovirus vector, the dosage for administration of adenovirus to humans can range from about 10⁶ to 10⁹ plaque forming unit (pfu) per injection but can be as high as 10¹² pfu per injection. Ideally, a subject will receive a single injection. If additional injections are necessary, they can be repeated at six-month intervals for an indefinite period and/or until the efficacy of the treatment has been established. Furthermore, the dosage can be adjusted according to the typical dosage for the specific disease or condition to be treated. Often a single dose can be sufficient; however, the dose can be repeated if desirable. The dosage should not be so large as to cause adverse side effects. Generally, the dosage will vary with the age, condition, sex and other parameters and can be determined by one of skill in the art according to routine methods (see e.g., Remington's Pharmaceutical Sciences). The individual physician in the event of any complication can also adjust the dosage.

The composition of this invention can typically include an effective amount of the nucleic acid of this invention in combination with a pharmaceutically acceptable carrier and, in addition, may include other medicinal agents, pharmaceutical agents, carriers, adjuvants, diluents, etc. By pharmaceutically acceptable is meant a material that is not biologically or otherwise undesirable, i.e., the material may be administered to an individual along with the selected agent without causing any undesirable biological effects or interacting in a deleterious manner with any of the other components of the pharmaceutical composition in which it is contained.

The compositions of the present invention can also be used in combination as multivalent vaccines in order to confer simultaneous protection against infection by more than one species or strain RSV.

In the present invention, the subject can be any mammal, preferably human, and can include but is not limited to mouse, rat, cow, guinea pig, hamster, rabbit, cat, dog, goat, sheep, monkey, horse and chimpanzee.

In the present invention, the modes of administration include oral administration, nebulization, inhalation, mucosal administration, intranasal administration, intratracheal administration, intravenous administration, intraperitoneal administration, subcutaneous administration and intramuscular administration.

The present invention is more particularly described in the following examples that are intended as illustrative only since numerous modifications and variations therein will be apparent to those skilled in the art.

### EXAMPLES

In this invention, the immune response in BALB/c mice intranasally immunized with MAA conjugated to polyethyleneimine (MP) bound to a DNA-F glycoprotein vaccine was studied. RSV challenge of mice vaccinated with a plasmid encoding RSV-F conjugated to MP (MAA-F) elicited both Th1 and Th2 cytokine responses with minimal inflammation in the lungs. CD3+ T lymphocytes from the lungs of mice vaccinated with MAA-F, but not the control MAA carrier, responded to RSV infection with rapid, but transient increases in IL-4 and IFNγ expression. Fewer cells infiltrated the lungs of MAA-F-vaccinated mice compared to RSV-vaccinated mice. These data show that MAA is a useful carrier to deliver nucleic acids to the site of RSV infection (lungs) and is effective in producing and/or enhancing RSV immunity.

Animals

Four- to five-week old, specific pathogen-free, female BALB/c mice were purchased from Jackson Laboratories (Bar Harbor, Me.), and fed water and food ad libitum.

Virus stock

RSV strain A2 (RSV/A2) was propagated in Vero cells (African green monkey kidney fibroblasts, ATCC CCL 81) as previously described (60). Virus titers were determined by plaque assay on Vero cells and immunostaining with anti-F (clone 130-8F) and -G (clone 131-2G) monoclonal antibodies as previously described (60, 61).

MAA particle preparation

MAA particles were prepared as previously described [50]. All reactions were preformed at room temperature unless otherwise indicated. 1 mg N-succinimidyl 3-(2-pyridyldithio)propionate (SPDP, Pierce, Rockford, Ill.) in 40 μl dimethylsulfoxide (DMSO, Sigma, St. Louis, Mo.) was added to 50 mg mouse serum albumin diluted in 1 ml of 0.1 M NaHCO₃ buffer, pH 8. The MAA-SPDP solution was mixed 4 h, and then filtered through a NAP-10 column (Pharmacia Biotech, Piscataway, N.J.). A 50% w/v solution of polyethyleneimine (PEI, Sigma) in water (pH 8) was added to 0.25 mg of SPDP diluted in DMSO, and mixed 4 h. The pH of the PEI solution was adjusted to 7.0, and 20 mg reductacryl (Calbiochem, La Jolla, Calif.) added and stirred 30 min. The resulting solution, PEI-SPDP, was purified by filtration over a NAP-10 column. MAA-SPDP and PEI-SPDP solutions were combined (MAA-PEI), mixed overnight, and the pH adjusted to 5.5-6.0. MAA-PEI particles were vigorously stirred on a heated hot plate until particle formation was evident. The beaker was cooled and the pH adjusted to 7.0 with 2M NaOH.

To determine the amount of PEI sites available for plasmid DNA binding, 1 μg plasmid DNA was added to different dilutions of MAA-PEI particles in a constant volume. The mixtures were incubated 20 min, and 4 μl of each dilution was fractionated on a 0.6% agarose gel. The lane at which no plasmid entered the gel represented the dilution of particles where the ratio of PEI to plasmid DNA was 2:1, the point at which all charges of the PEI are neutralized [50].

Vaccine and challenge

RSV F glycoprotein DNA vaccine (pDNA-F) was prepared using pcDNAneo 3 vector plasmid (Invitrogen, San Diego, Calif.) constructed with RSV F glycoprotein cDNA from RSV/A2 [62]. The RSV F glycoprotein nucleic acid sequence utilized to prepare an RSV-F glycoprotein DNA vaccine is provided herein as SEQ ID NO: 1. pDNA-F was propagated in E. coli SURE2 cells (Stratagene, La Jolla, Calif.) and purified using an EndoFree Plasmid Giga Kit (Qiagen, Valencia, Calif.) following the manufacturer's instructions.

MAA-PEI particles were incubated with PBS as a control (MAA), or with 1 μg or 10 μg pDNA-F (MAA-F), for 20 min at room temperature to prepare the vaccines. Mice were anesthetized with Avertin (2,2,2-tribromoethanol), intranasally immunized with 1 or 10 μg of MAA or MAA-F in 50 μl PBS/mouse, and rested for 2 weeks prior to RSV/A2 challenge or vaccine boost. A group of the mice receiving a single vaccination (1X) of 1 μg MAA or MAA-F vaccine were i.n. challenged with 2×10⁵ pfu of RSV/A2 (day 0 for 1X vaccinated mice), and the remainder i.n. boosted (2X) with an additional 1 μg of MAA or MAA-F. Boosted mice receiving a total of 2 μg of MAA or MAA-F vaccine were rested 2 weeks, and then challenged with 10⁶ pfu of RSV/A2 (day 0 for 2X vaccinated mice). Mice receiving 10 μg of MAA or MAA-F vaccine were rested 2 weeks, and then challenged with 10⁶ pfu of RSV/A2. Preliminary experiments examining mice vaccinated 1X with 1 μg MAA-F showed limited immune enhancement, therefore these mice were challenged with a lower dose (2 ×10⁵ pfu) of RSV/A2. Preliminary experiments examining 2X MAA-F vaccinated mice, or mice vaccinated 1X with 10 μg MAA-F, suggested that higher dose vaccination substantially improved immune responses. Therefore, to better understand immune correlates of protection and their relationship to previous RSV studies in the BALB/c mouse model [75, 76], mice vaccinated 1X with 10 μg vaccine or 2X with a total of 2 μg of vaccine were challenged with 10⁶ pfu of RSV/A2.

Cell sampling

At days 0, 4, 12 or 14 post-RSV challenge of DNA vaccinated mice, 6 mice/treatment were anesthetized with Avertin, exsanguinated by severing the right caudal artery, and blood specimens collected for sera. All procedures were performed on ice. Bronchoalveolar leukocytes (BAL) were collected by lavage using three 1 ml washes of PBS (GIBCO Life Technologies), washed, and resuspended in PBS containing 1% bovine serum albumin (Sigma). The lungs were removed and stored at −70° C. until use. The spleens were collected in Hanks balanced salt solution (HBSS, GIBCO Life Technologies), dissociated using a glass pestle and 100 μm cell strainer (Falcon, Franklin Lakes, N.J.), washed, and resuspended in Dulbecco's Modified Eagle suspension medium (sDME, GIBCO Life Technologies) containing 10% heat-inactivated fetal bovine serum (Hyclone, Logan, Utah). The total number of leukocytes recovered from naïve, unvaccinated mice was <5,500 cells/lung.

Flow cytometry

All flow cytometry procedures were done on ice. BAL and spleen cells were blocked with 10% normal mouse sera (The Jackson Laboratory, Bar Harbor, Me.) diluted in PBS for 30 min. The phenotypes of the cells were determined as previously described (60, 61). Briefly, the cell subsets were determined by staining with appropriate dilutions of phycoerythrin (PE)- or fluorescein isothiocyanate (FITC)-conjugated anti-CD3ε (145-2C11), anti-CD45R/B220 (RA3-6B2), anti-pan NK cell (DX-5), anti-neutrophil, Ly-6G (RB6-8C5), anti-integrin molecule, CD11b (M1/70), anti-CD4 (LM4-4), and anti-CD8 (53-6.7) monoclonal antibodies (Pharmingen, San Diego, Calif.) diluted in D-PBS containing 1% BSA (Sigma, St. Louis, Mo.). Intracellular cytokine staining was examined directly ex vivo as previously described (61). Briefly, intracellular cytokine transport from the Golgi apparatus was inhibited by incubating the cells for 4 hr at 37° C. in PBS containing GolgiStop (PharMingen). The cells were washed in PBS and cell surface stained with FITC-conjugated anti-CD3ε antibody for 30 min at 4° C. Washed cells were fixed and permeabilized with Cytofix/Cytoperm buffer (PharMingen) for 30 min at 4° C. and subsequently stained with PE-labeled anti-interleukin-2 (IL-2)(JES6-5H4), anti-IL-4 (BVD4-1D11), anti-IL5 (TRFK5), anti-IL-10 (JES5-16E3), anti-TNFα (MP6-XT22) or anti-gamma interferon (IFNγ)(XMG1.2) monoclonal antibody diluted in PBS containing 1% BSA and 0.1% saponin for 30 min at 4° C. The cells were washed and resuspended in D-PBS containing 1% BSA for flow cytometric analysis (FACScan, Becton Dickinson, Palo Alto, Calif.).

Hematoxylin and Eosin (H&E) staining

To further differentiate BAL cell subsets, BAL cells diluted in PBS containing 1% BSA were cytospun (Shandon, Pittsburgh, Pa.) onto glass microscope slides, fixed, and stained with hematoxylin and eosin (H&E; Sigma, St. Louis, Mo.) as described previously (61).

Anti-RSV-F serum antibody ELISA

Serum was analyzed for RSV anti-F antibodies using a modified ELISA protocol (63). Briefly, microtiter plates (Costar, Cambridge, Mass.) were coated with 5 ng/ml purified F glycoprotein isolated from RSV-infected Vero cells (64), or uninfected Vero cell lysate in bicarbonate buffer. Dilutions of sera in PBS were added to triplicate wells and incubated 1 hr at 37° C. The plates were washed 3× with PBS containing 0.05% Tween (washing buffer), and incubated 1 hr at 37° C. with horseradish peroxidase-conjugated goat anti-mouse immunoglobulin (Accurate Chemical, Westbury, N.Y.). The plates were washed 3× with washing buffer and developed with 3, 3′, 5, 5′-tetramethylbenzidine (TMB, Sigma, St. Louis, Mo.) as described by the manufacturer. The difference between the absorbance readings of wells coated with purified RSV-F and wells coated with uninfected cell lysate were used to determine the RSV-specific antibody titer.

Serum was also examined for levels of neutralizing antibodies. Serum samples were diluted at 1:20, 1:40 and 1:80 in serum-free DMEM, incubated with 10³ pfu RSV/A2 for 1 hr at 37° C., and then added to subconfluent Vero cells in 24-well flat bottom plates (Costar, Cambridge, Mass.). The plates were incubated for 2 hr at 37° C., the overlay removed, and replaced with DMEM containing 10% FBS. The plates were incubated for 3 days at 37° C., the overlay removed, the wells carefully washed with PBS, and the cells fixed in 80% acetone/20% PBS. Plaques were enumerated by immunostaining with anti-F (130-8F) and anti-G (131-2G) monoclonal antibodies as previously described [61]. Appropriately diluted guinea pig hyperimmune anti-RSV F serum was used as a positive control for inhibition of plaque formation, and naive mouse serum was used as a negative control.

Statistical analysis

For each experiment, 6 individual animals per vaccine group/time point were examined. The data shown represents the average ± standard deviation from a representative experiment. Significant differences were determined by unpaired two-tail analysis and are indicated if p<0.05.

ELISPOT assay

A modified ELISPOT assay was used to determine the frequency of anti-RSV antibody-expressing spleen cells as previously described (65). Briefly, multiscreen-HA 96-well microtiter plates (Millipore, Bedford, Mass.) were coated with RSV-infected or uninfected Vero cell lysate in PBS and incubated at 37° C. for 1 h. The coated plates were blocked at room temperature with PBS containing 2% fetal calf serum (Hyclone, Logan, Utah). Prior to addition of the spleen cells, red blood cells were removed by hypotonic lysis (9 volumes of 0.83% w/v NH₄Cl in water and 1 volume of 2.06% w/v Tris in water, pH 7.65; pH to 7.2). 10⁶ spleen cells/well were added to the plates and incubated 4 hr at 37° C. The plates were washed once with PBS, once with wash buffer, and 3× with PBS. Alkaline phosphatase-conjugated goat-anti-mouse IgG (Boehringer-Mannheim, Indianapolis, Ind.) was added to the wells and the plates incubated overnight at room temperature. The plates were washed as described above and enumerated using 5-bromo-4-chloro-3-inolyl phosphate (Sigma) dissolved to 1 mg/ml in substrate buffer (0.1M Tris-Cl, pH 9.5; 10% v/v diethanolamine; 0.1M NaCl; 5 mM MgCl₂). The frequency of RSV-specific antibody-secreting cells was determined from the number of ELISPOTS in wells coated with RSV-infected Vero lysate minus the number of ELISPOTS in wells coated with uninfected Vero lysate over the cell input/well.

Viral lung titers

Lungs collected at various days pi were analyzed for virus titer as described previously (61). Briefly, the lungs were homogenized in PBS containing 30% sucrose (Sigma). The virus titers in the homogenates were determined by plaque assay on Vero cell monolayers grown in 24-well flat-bottom tissue culture plates (Costar, Cambridge, Mass.). Ten-fold dilutions of the homogenates, diluted in serum-free DMEM were added to the wells and incubated for 2 hr at 37° C. The overlay containing the homogenate was removed and replaced with DMEM containing 10% FCS. The plates were incubated between 4-6 days at 37° C. until detectable CPE was observed. The overlay was removed, the wells washed carefully with PBS, and fixed in 80% acetone/20% PBS. Plaques were enumerated by immunostaining with anti-F (130-8F) and anti-G (131-2G) monoclonal antibodies as previously described (61).

Virus load is reduced by higher dose MAA-F vaccination

The virus titers in the lungs of MAA or MAA-F vaccinated mice challenged with RSV/A2 were determined as a measure of vaccine efficacy. Peak virus titers for any vaccination occurred at day 4 pi. No significant difference in virus titers were observed in mice vaccinated 1X with 1 μg MAA-F (2.9-3.4 log PFU/g lung tissue) compared to control MAA vaccinated mice (3.1-3.4 log PFU/g lung tissue; p=0.34). In contrast, mice vaccinated with higher doses of MAA-F had significantly reduced virus titers at day 4 pi compared to MAA controls. At day 4 pi, virus titers were significantly lower in 2X MAA-F vaccinated mice (3.7-4.3 log PFU/g lung tissue) compared to 2X MAA vaccinated mice (4.9-5.1 log PFU/g lung tissue; p=0.007), and significantly lower in mice vaccinated 1X with 10 μg MAA-F (2.9-3.5 log PFU/g lung tissue) compared to mice vaccinated 1X with 10 μg MAA (3.6-4.1 PFU/g lung tissue, p=0.017). As expected, no virus was detected at day 12 pi in MAA or MAA-F vaccinated mice, since RSV is generally cleared by day 10 pi in BALB/c mice [40]. These data show that lung-targeted DNA vaccination with 2 or 10 μg of MAA-F is sufficient to enhance resistance to RSV/A2 challenge.

MAA-F vaccination and the anti-F glycoprotein antibody response.

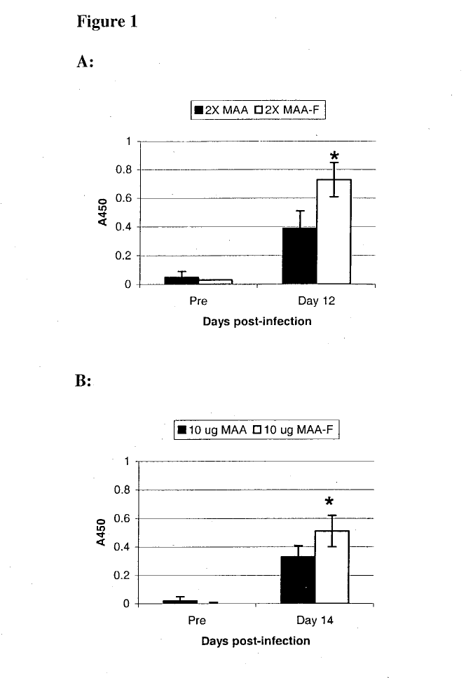

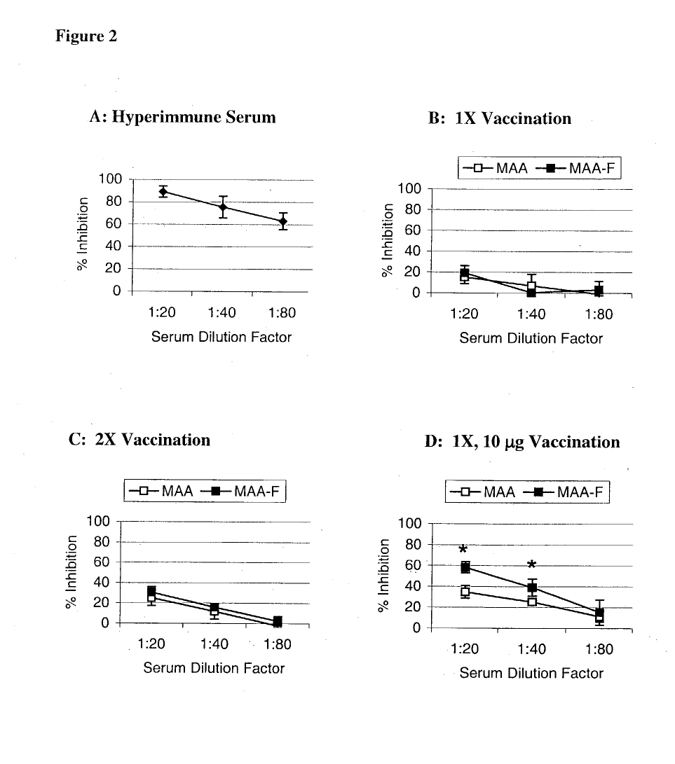

MAA has been shown to be a useful carrier to target DNA vaccination of lung macrophage and dendritic cells [55], and dendritic cell-targeted vaccination has been shown to induce primary IgG responses as early as 5 days post-vaccination [56]. The serum anti-F glycoprotein antibody response was examined at day 12 or 14 post-RSV/A2 challenge of mice vaccinated 1X with 1 μg or 10 μg MAA or MAA-F, or 2X MAA or MAA-F vaccination (FIG. 1). No significant anti-F glycoprotein antibody response was detected at day 0, 12 or 14 post-RSV/A2 challenge of 1X MAA, or MAA-F vaccinated mice. Prior to RSV/A2 challenge, low levels of anti-F glycoprotein antibodies were detected in 2X MAA and MAA-F vaccinated mice (FIG. 1A) and in mice vaccinated 1X with 10 μg MAA or MAA-F (FIG. 1B), however at days 12 or 14 post-RSV/A2 challenge, increased levels of anti-F glycoprotein antibody were observed for all vaccine treatments. Significantly higher anti-F glycoprotein antibody responses occurred in 2X MAA-F vaccinated mice and in mice vaccinated 1X with 10 μg MAA-F, compared to 2X MAA vaccinated mice or mice vaccinated 1X with 10 μg MAA. Interestingly, mice vaccinated 1X with 10 μg MAA-F had higher total anti-RSV antibody responses at day 14 post-RSV/A2 challenge compared to 1X or 2X MAA-F vaccinated, or MAA control vaccinated mice. Consistent with these findings, mice vaccinated 1X with 10 μg MAA-F had higher serum neutralizing antibodies than 1X or 2X MAA-F vaccinated, or MAA control vaccinated mice (FIG. 2). These results suggest that higher doses of MAA-F vaccine increase the anti-F glycoprotein antibody response and are associated with enhanced virus clearance.

To further evaluate the humoral response, the total number of anti-RSV antibody-secreting cells (ASCs) in the spleens of vaccinated mice was determined following RSV/A2 challenge. The total number of anti-RSV ASCs in 2X MAA-F vaccinated mice was 3.8-fold higher at day 4 post-challenge (p=0.0008), and 2.6-fold higher at day 12 post-challenge (p=0.008), compared to 2X MAA vaccinated mice. Similarly, the total number of anti-RSV ASCs in spleen cells from mice vaccinated 1X with 10 μg MAA-F was 3.2-fold higher at day 4 post-challenge (p=0), and 3.6-fold at day 14 post-challenge (p=0), compared to mice vaccinated 1X with 10 μg MAA. These results are consistent with the increased anti-F glycoprotein antibody response and enhanced virus clearance at day 4 pi in mice vaccinated with 2 or 10 μg MAA-F compared to control MAA vaccinated mice.

MAA-F vaccination and cytokine expression.

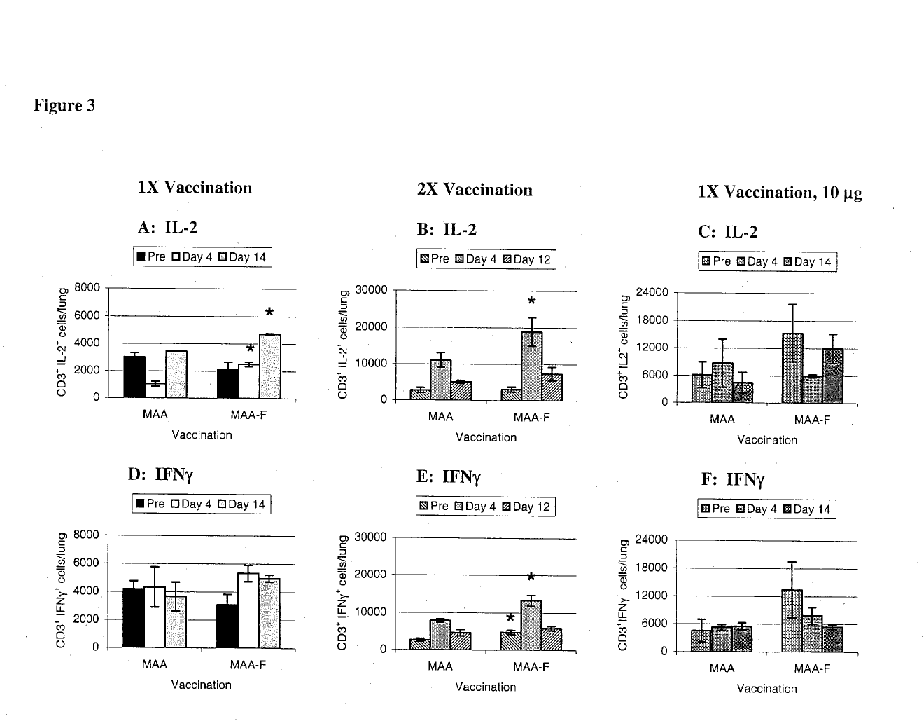

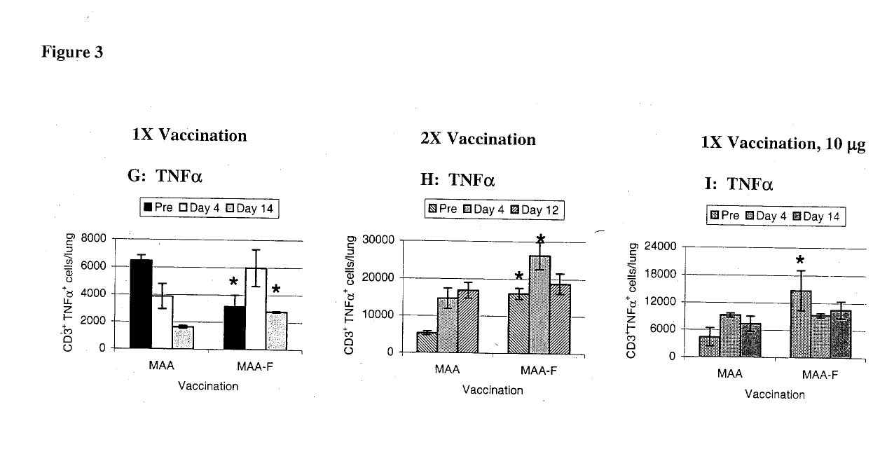

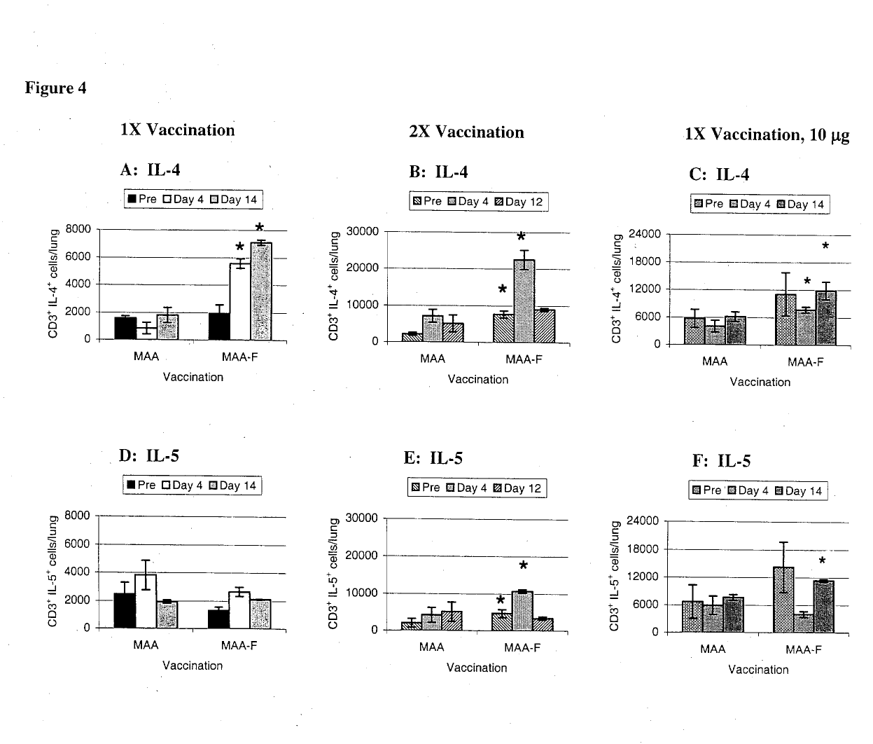

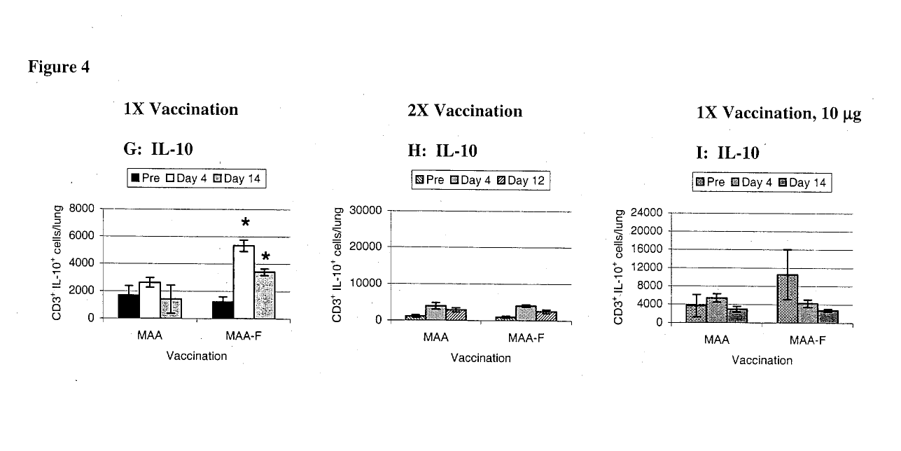

The ex vivo pattern of intracellular Th1- or Th2-type cytokine expression by CD3⁺ BAL cells from MAA or MAA-F vaccinated mice challenged with RSV/A2 was determined (FIGS. 3 and 4). The number of CD3⁺ BAL cells expressing IL-4 or IL-10 was 3- to 4-fold higher in 1X MAA-F vaccinated mice compared to 1X MAA vaccinated mice, as were the number of CD3⁺ BAL cells expressing IL-2. No significant differences in the numbers of cells expressing IFNγ or IL-5 were observed in 1X MAA or MAA-F vaccinated mice. In contrast, the number of CD3⁺ BAL cells which expressed IL-2, IFNγ, TNFα, IL-4 or IL-5 was ˜3-fold higher in 2X MAA-F vaccinated mice compared to 2X MAA vaccinated mice. Similar to 2X MAA-F vaccinated mice, the number of CD3⁺ BAL cells expressing TNFα, IL-4 or IL-5 were higher in mice vaccinated 1X with 10 μg MAA-F compared to control MAA vaccinated mice. These results indicate that induction of Th1- and/or Th2-type cytokine responses may be influenced by the number of MAA-F vaccinations, or by the level of F glycoprotein expression.

MAA -F vaccination and pulmonary inflammation.

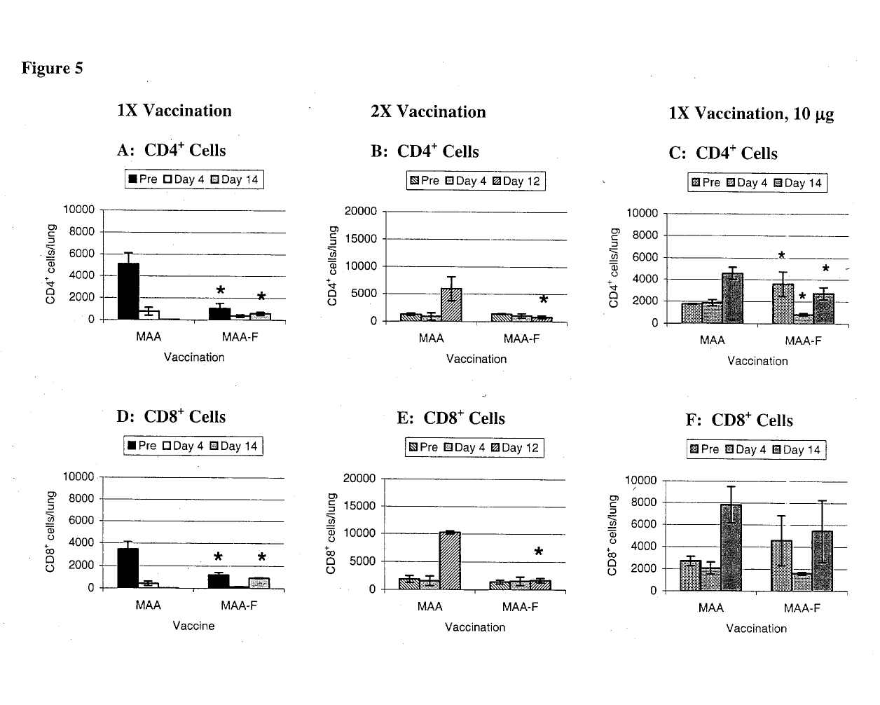

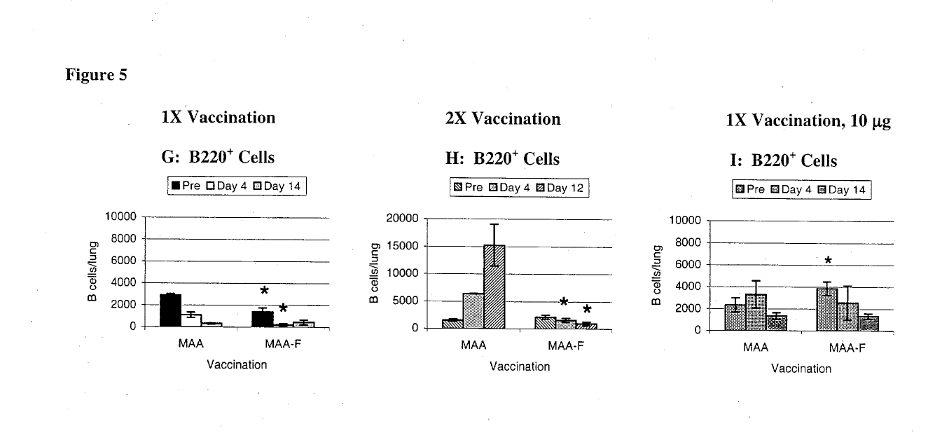

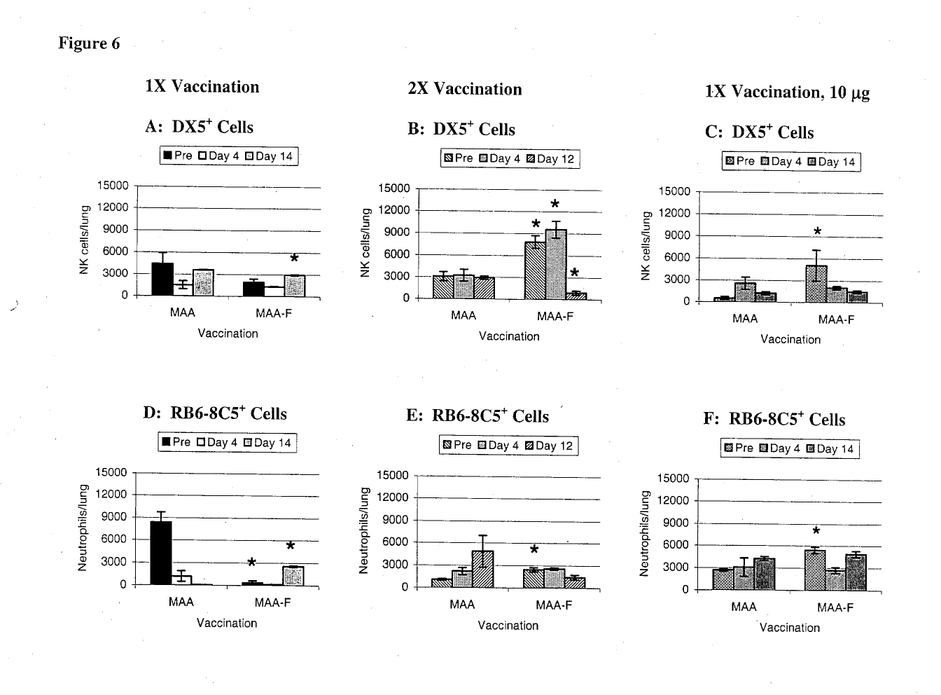

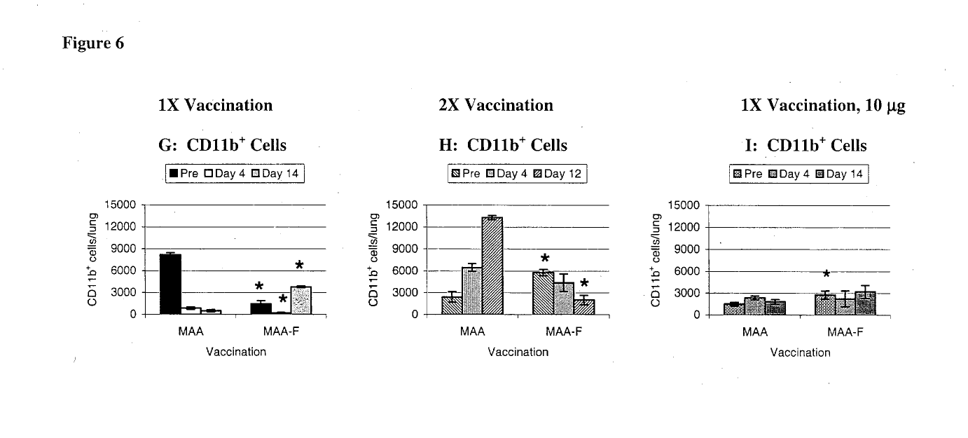

MAA is typically used in lung scanning procedures without inducing significant pulmonary inflammation [52-54]. To evaluate the pulmonary cellular response to MAA or MAA-F vaccination, the total number of CD4⁺, CD8⁺, B220⁺, DX5⁺, RB6-8C5⁺, or CD11b⁺ BAL cell types present in the lung after vaccination or RSV/A2 challenge were determined (FIGS. 5 and 6). Higher numbers of CD4⁺, CD8⁺ B220⁺, RB6-8C5⁺ and CD11b+ cells were in the lungs of 1X MAA vaccinated mice prior to RSV/A2 challenge compared to day 4 or 14 post-challenge. Mice vaccinated with 1X MAA-F had low numbers of all cell types prior to RSV challenge, but slightly more DX5⁺ cells, RB6-8C5⁺ and CD11b⁺ cells after RSV challenge, suggesting that 1X MAA-F vaccination does not induce substantial pulmonary cellular inflammation. In contrast to 1X MAA vaccination, lower numbers of all cell types were observed in 2X MAA vaccinated mice and mice vaccinated 1X with 10 μg MAA prior to RSV challenge, however all BAL cell types increased at days 12 or 14 post-RSV challenge. No remarkable differences in CD4⁺, CD8⁺, B220⁺, or RB6-8C5⁺ cell numbers were observed before or after RSV challenge of 2X MAA-F vaccinated mice, however higher numbers of DX5⁺ cells were evident at day 4 post-challenge. These results suggest that 2X MAA-F vaccination is not associated with substantial pulmonary cellular inflammation, and that 2X MAA-F vaccination may augment pulmonary NK cell trafficking, an effect that may contribute to enhanced virus clearance observed at day 4 pi. Although the DX5 antibody is commonly used as a pan NK cell marker, the antibody reacts with CD49B (α2 integrin) that is expressed at lower levels on CD8⁺ T cell. Thus, it is possible that the increased number of DX5⁺ cells observed in 2X MAA-F vaccinated mice challenged with RSV/A2 may represent both NK cells and CD8⁺ cells. No significant differences in CD8⁺, B220⁺, DX5⁺, RB6-8C5⁺ or CD11b⁺ cell numbers were observed in mice vaccinated 1X with 10 μg MAA-F compared to control MAA vaccinated mice, however fewer numbers of CD4⁺ cells were observed in MAA-F compared to control MAA vaccinated mice. For most cell types examined, higher numbers of cells trafficked to the lungs of mice vaccinated 1X with 10 μg MAA or MAA-F compared to mice vaccinated 1X with 1 μg MAA or MAA-F. These results indicate that higher doses of MAA or MAA-F are associated with increased pulmonary cellular infiltrate.

Hematoxylin and Eosin (H&E) differentiation of BAL cells

The percentages of lymphocytes, macrophages, polymorphonuclear (PMN) cells, and eosinophils in the BAL following RSV challenged of DNA vaccinated mice was examined by hematoxylin and eosin staining (Table 1). Few to no eosinophils were detected in MAA or MAA-F vaccinated mice. Macrophages were the predominant cell type in the BAL of 1X MAA or MAA-F vaccinated mice, and notably higher in mice vaccinated 1X with 10 μg of MAA-F prior to RSV/A2 challenge. 2X MAA and MAA-F vaccinated mice and mice vaccinated 1X with 10 μg MAA or MAA-F had higher percentages of pulmonary lymphocytes, with the highest percentage occurring between days 4 and 12 pi post-challenge, compared to 1X MAA or MAA-F vaccinated mice. Interestingly, higher percentages of PMN were detected in the BAL from 2X MAA or MAA-F vaccinated mice compared to all other vaccinations. These results suggest that the pulmonary cellular response to MAA or MAA-F vaccination is similar prior to RSV/A2 challenge, however higher dosage or repeated MAA-F vaccination may induce a greater pulmonary lymphocyte response.

Features of the humoral and cellular immune response associated with pulmonary vaccination using a novel DNA vaccine carrier, MAA, conjugated to an RSV DNA vaccine, MAA-F were examined. This invention shows that low dose vaccination, i.e. 2 or 10 μg of plasmid DNA encoding the RSV F glycoprotein, enhances the serum anti-F glycoprotein antibody response, patterns of cytokine expression, and virus clearance following RSV challenge. The reduced virus titers associated with 2 or 10 μg vaccination with MAA-F, but not 1 μg MAA-F, suggests that the threshold for developing a protective response may be related to the quantity or schedule of MAA-F vaccine administration. There was not a clear association between Th1- or Th2-type cytokine responses, or the response by pulmonary cell subsets 1X or 2X MAA-F vaccinated mice following RSV/A2 challenge, suggesting that the DNA vaccine carrier, MAA, may have adjuvant-like effects.

Adjuvants are commonly used to promote aspects of the immune response to vaccination. MAA has previously been shown to be an effective mucosal DNA vaccine delivery agent that accumulates in the alveolar interstitium without inducing significant inflammation, and targets pulmonary interstitial macrophages and dendritic cells [52-54]. The adjuvant-like effects associated with MAA in this study may be associated with activation of pulmonary dendritic cells during particle uptake. It is also possible that the DNA plasmid vector used in these studies has adjuvant-like effects. pcDNAneo has been shown to contain immune stimulatory CpG motifs [77], which may promote cytokine responses [78,79]. CpG motifs may also act as potent adjuvants for F glycoprotein vaccination, an effect previously shown to enhance the anti-RSV F glycoprotein humoral response [80].

Consistent with the adjuvant-like effects of MAA, hematoxylin and eosin (H&E) differentiation and cell surface analysis of cell types in the BAL showed no consistent differences in cell types associated with MAA or MAA-F vaccination. However, a higher percentage of lymphocytes in the lungs were indicated by H&E differentiation of BAL cells from mice vaccinated 1X with 10 μg MAA and MAA-F and in 2X MAA or MAA-F vaccinated mice, and higher numbers of DX5⁺ cells were evident at day 4 post-challenge in 2X MAA-F vaccinated mice. The increased numbers of DX5⁺ cells likely represent a majority of NK cells, however a portion of DX5⁺ cells may include CD8⁺T cells, as the DX5 antibody recognizes CD49B that is expressed at higher levels on NK cells and lower levels on CD8⁺ T cell. The higher lymphocyte number detected in the BAL may represent the residual response to MAA particles. The BAL cell numbers in rested 1X or 2X MAA vaccinated mice (range 1.1-1.9 X 10⁵ cells/mouse) or 1X or 2X MAA-F vaccinated mice (range 1.3-1.7 X 10⁵ cells/mouse) were higher prior to RSV challenge compared to BAL cell numbers in naive mice (0.5-1.2×10⁵ cells/mouse).

Increased host resistance to pathogen challenge is one measure of vaccine efficacy, an effect achieved by 2X MAA-F or 1X vaccination with 10 μg MAA-F, and shown by enhanced virus clearance following RSV challenge. Immune correlates of host resistance that were associated were increased anti-F glycoprotein serum antibody responses, and increased frequencies of anti-RSV antibody secreting cells. Interestingly, mice vaccinated with 10 μg MAA-F also had increased levels of anti-RSV neutralizing antibodies following RSV challenge. Thus, this invention shows that MAA can be an effective carrier for delivery of a RSV DNA vaccine expressing RSV F glycoprotein, and shows that pulmonary targeting can be a useful method for targeting DNA vaccines directed against other respiratory pathogens.

Throughout this application, various publications are referenced. The disclosures of these publications in their entireties are hereby incorporated by reference into this application in order to more fully describe the state of the art to which this invention pertains.

### References

1. Hall, C. B., J. M. Geiman, R. G. Douglas, Jr., and M. P. Meagher. 1978. Control of nosocomial respiratory syncytial viral infections. Pediatrics 62:728.

2. Hall, C. B., E. E. Walsh, C. E. Long, and K. C. Schnabel. 1991. Immunity to and frequency of reinfection with respiratory syncytial virus. J Infect. Dis. 163:693.

3. Hall, C. B. 1999. Respiratory syncytial virus: A continuing culprit and conundrum. J. Pediatr. 135:2.

4. Crowe, J. E., Jr. 1995. Current approaches to the development of vaccines against disease caused by respiratory syncytial virus (RSV) and parainfluenza virus (PIV). A meeting report of the WHO Programme for Vaccine Development. Vaccine 13:415.

5. Falsey, A. R., J. J. Treanor, R. F. Betts, and E. E. Walsh. 1992. Viral respiratory infections in the institutionalized elderly: clinical and epidemiologic findings. J Am. Geriatr. Soc. 40:115.

6. Henderson, F. W., A. M. Collier, W. A. Clyde, Jr., and F. W. Denny. 1979. Respiratory-syncytial-virus infections, reinfections and immunity. A prospective, longitudinal study in young children. N. Engl. J. Med. 300:530.

7. Beem, M. 1967. Repeated infections with respiratory syncytial virus. J lmmunol. 98:1115.

8. Kim, H. W., J. G. Canchola, C. D. Brandt, G. Pyles, R. M. Chanock, K. Jensen, and R. H. Parrott. 1969. Respiratory syncytial virus disease in infants despite prior administration of antigenic inactivated vaccine. Am. J. Epidemiol. 89:422.

9. Kapikian, A. Z., R. H. Mitchell, R. M. Chanock, R. A. Shvedoff, and C. E. Stewart. 1969. An epidemiologic study of altered clinical reactivity to respiratory syncytial (RS) virus infection in children previously vaccinated with an inactivated RS virus vaccine. Am. J Epidemiol. 89:405.

10. Fulginiti, V. A., J. J. Eller, O. F. Sieber, J. W. Joyner, M. Minamitani, and G.

Meiklejohn. 1969. Respiratory virus immunization. I. A field trial of two inactivated respiratory virus vaccines; an aqueous trivalent parainfluenza virus vaccine and an alum-precipitated respiratory syncytial virus vaccine. Am. J. Epidemiol. 89:435.

11. Chin, J., R. L. Magoffin, L. A. Shearer, J. H. Schieble, and E. H. Lennette. 1969. Field evaluation of a respiratory syncytial virus vaccine and a trivalent parainfluenza virus vaccine in a pediatric population. Am. J. Epidemiol. 89:449.

12. Connors, M., N. A. Giese, A. B. Kulkarni, C. Y. Firestone, H. C. Morse, 3rd, and B. R. Murphy. 1994. Enhanced pulmonary histopathology induced by respiratory syncytial virus (RSV) challenge of formalin-inactivated RSV-immunized BALB/c mice is abrogated by depletion of interleukin-4 (IL-4) and IL-10. J. Virol. 68:5321.

13. Waris, M. E., C. Tsou, D. D. Erdman, S. R. Zaki, and L. J. Anderson. 1996. Respiratory synctial virus infection in BALB/c mice previously immunized with formalin-inactivated virus induces enhanced pulmonary inflammatory response with a predominant Th2-like cytokine pattern. J. Virol. 70:2852.

14. Graham, B. S., G. S. Henderson, Y. W. Tang, X. Lu, K. M. Neuzil, and D. G. Colley. 1993. Priming immunization determines T helper cytokine mRNA expression patterns in lungs of mice challenged with respiratory syncytial virus. J. Immunol. 151:2032.

15. Hussell, T., L. C. Spender, A. Georgiou, A. O'Garra, and P. J. Openshaw. 1996. Th1 and Th2 cytokine induction in pulmonary T cells during infection with respiratory syncytial virus. J. Gen. Virol. 77 (Pt 10):2447.

16. Srikiatkhachorn, A., and T. J. Braciale. 1997. Virus-specific memory and effector T lymphocytes exhibit different cytokine responses to antigens during experimental murine respiratory syncytial virus infection. J Virol. 71:678.

17. Boelen, A., A. Andeweg, J. Kwakkel, W. Lokhorst, T. Bestebroer, J. Dormans, and T. Kimman. 2000. Both immunisation with a formalin-inactivated respiratory syncytial virus (RSV) vaccine and a mock antigen vaccine induce severe lung pathology and a Th2 cytokine profile in RSV-challenged mice. Vaccine 19:982.

18. Bangham, C. R. 1986. Passively acquired antibodies to respiratory syncytial virus impair the secondary cytotoxic T-cell response in the neonatal mouse. Immunology 59:37.

19. Crowe, J. E., Jr., C. Y. Firestone, and B. R. Murphy. 2001. Passively acquired antibodies suppress humoral but not cell-mediated immunity in mice immunized with live attenuated respiratory syncytial virus vaccines. J. Immunol. 167:3910.

20. Englund, J. A. 1994. Passive protection against respiratory syncytial virus disease in infants: the role of maternal antibody. Pediatr. Infect. Dis. J. 13:449.

21. Karron, R. A., P. F. Wright, J. E. Crowe, Jr., M. L. Clements-Mann, J. Thompson, M. Makhene, R. Casey, and B. R. Murphy. 1997. Evaluation of two live, cold-passaged, temperature-sensitive respiratory syncytial virus vaccines in chimpanzees and in human adults, infants, and children. J. Infect. Dis. 176:1428.

22. Kim, H. W., J. O. Arrobio, G. Pyles, C. D. Brandt, E. Camargo, R. M. Chanock, and R. H. Parrott. 1971. Clinical and immunological response of infants and children to administration of low-temperature adapted respiratory syncytial virus. Pediatrics 48:745.

23. Kim, H. W., J. O. Arrobio, C. D. Brandt, P. Wright, D. Hodes, R. M. Chanock, and R. H. Parrott. 1973. Safety and antigenicity of temperature sensitive (TS) mutant respiratory syncytial virus (RSV) in infants and children. Pediatrics 52:56.

24. Wright, P. F., T. Shinozaki, W. Fleet, S. H. Sell, J. Thompson, and D. T. Karzon. 1976. Evaluation of a live, attenuated respiratory syncytial virus vaccine in infants. J. Pediatr. 88:931.

25. Wright, P. F., R. B. Belshe, H. W. Kim, L. P. Van Voris, and R. M. Chanock. 1982. Administration of a highly attenuated, live respiratory syncytial virus vaccine to adults and children. Infect. Immun. 37:397.

26. Wright, P. F., R. A. Karron, R. B. Belshe, J. Thompson, J. E. Crowe, Jr., T. G. Boyce, L. L. Halburnt, G. W. Reed, S. S. Whitehead, E. L. Anderson, A. E. Wittek, R. Casey, M. Eichelberger, B. Thumar, V. B. Randolph, S. A. Udem, R. M. Chanock, and B. R. Murphy. 2000. Evaluation of a live, cold-passaged, temperature-sensitive, respiratory syncytial virus vaccine candidate in infancy. J Infect. Dis. 182:1331.

27. Barouch, D. H., S. Santra, K. Tenner-Racz, P. Racz, M. J. Kuroda, J. E. Schmitz, S. S. Jackson, M. A. Lifton, D. C. Freed, H. C. Perry, M. E. Davies, J. W. Shiver, and N. L. Letvin. 2002. Potent CD4+ T cell responses elicited by a bicistronic HIV-1 DNA vaccine expressing gp120 and GM-CSF. J Immunol. 168:562.

28. Gurunathan, S., D. M. Klinman, and R. A. Seder. 2000. DNA vaccines: immunology, application, and optimization*. Annu. Rev. Immunol. 18:927.

29. Johnson, P. A., M. A. Conway, J. Daly, C. Nicolson, J. Robertson, and K. H. Mills. 2000. Plasmid DNA encoding influenza virus haemagglutinin induces Th1 cells and protection against respiratory infection despite its limited ability to generate antibody responses. J. Gen. Virol. 81:1737.

30. McCluskie, M. J., C. L. Brazolot Millan, R. A. Gramzinski, H. L. Robinson, J. C. Santoro, J. T. Fuller, G. Widera, J. R. Haynes, R. H. Purcell, and H. L. Davis. 1999. Route and method of delivery of DNA vaccine influence immune responses in mice and non-human primates. Mol. Med. 5:287.

31. Operschall, E., T. Schuh, L. Heinzerling, J. Pavlovic, and K. Moelling. 1999. Enhanced protection against viral infection by co-administration of plasmid DNA coding for viral antigen and cytokines in mice. J. Clin. Virol. 13:17.

32. Narum, D. L., S. Kumar, W. O. Rogers, S. R. Fuhrmann, H. Liang, M. Oakley, A. Taye, B. K. Sim, and S. L. Hoffman. 2001. Codon optimization of gene fragments encoding Plasmodium falciparum merzoite proteins enhances DNA vaccine protein expression and immunogenicity in mice. Infect. Immun. 69:7250.

33. Cusi, M. G., R. Zurbriggen, M. Valassina, S. Bianchi, P. Durrer, P. E. Valensin, M. Donati, and R. Gluck. 2000. Intranasal immunization with mumps virus DNA vaccine delivered by influenza virosomes elicits mucosal and systemic immunity. Virology 277:111.

34. Dong-Ji, Z., X. Yang, C. Shen, H. Lu, A. Murdin, and R. C. Brunham. 2000. Priming with Chlamydia trachomatis major outer membrane protein (MOMP) DNA followed by MOMP ISCOM boosting enhances protection and is associated with increased immunoglobulin A and Th1 cellular immune responses. Infect. Immun. 68:3074.

35. Okada, E., S. Sasaki, N. Ishii, I. Aoki, T. Yasuda, K. Nishioka, J. Fukushima, J. Miyazaki, B. Wahren, and K. Okuda. 1997. Intranasal immunization of a DNA vaccine with IL-12- and granulocyte-macrophage colony-stimulating factor (GM-CSF)-expressing plasmids in liposomes induces strong mucosal and cell- mediated immune responses against HIV-1 antigens. J. Immunol. 159:3638.

36. McCluskie, M. J., Y. Chu, J. L. Xia, J. Jessee, G. Gebyehu, and H. L. Davis. 1998. Direct gene transfer to the respiratory tract of mice with pure plasmid and lipid-formulated DNA. Antisense Nucleic Acid Drug Dev. 8:401.

37. Heeg, K., and S. Zimmermann. 2000. CpG DNA as a Th1 trigger. Int. Arch. Allergy Immunol. 121:87.

38. Krieg, A. M., and J. N. Kline. 2000. Immune effects and therapeutic applications of CpG motifs in bacterial DNA. Immunopharmacology 48:303.

39. Tripp, R. A., L. Jones, L. J. Anderson, and M. P. Brown. 2000. CD40 ligand (CD154) enhances the Th1 and antibody responses to respiratory syncytial virus in the BALB/c mouse. J. Immunol. 164:5913.

40. Li, X., S. Sambhara, C. X. Li, L. Ettorre, I. Switzer, G. Cates, O. James, M. Parrington, R. Oomen, R. P. Du, and M. Klein. 2000. Plasmid DNA encoding the respiratory syncytial virus G protein is a promising vaccine candidate. Virology 269:54.

41. Bembridge, G. P., N. Rodriguez, R. Garcia-Beato, C. Nicolson, J. A. Melero, and G. Taylor. 2000. DNA encoding the attachment (G) or fusion (F) protein of respiratory syncytial virus induces protection in the absence of pulmonary inflammation. J. Gen. Virol. 81:2519.

42. Lambert, D. M., and M. W. Pons. 1983. Respiratory syncytial virus glycoproteins. Virology 130:204.

43. Connors, M., P. L. Collins, C. Y. Firestone, and B. R. Murphy. 1991. Respiratory syncytial virus (RSV) F, G, M2 (22K), and N proteins each induce resistance to RSV challenge, but resistance induced by M2 and N proteins is relatively short-lived. J. Virol. 65:1634.

44. Power, U. F., T. N. Nguyen, E. Rietveld, R. L. de Swart, J. Groen, A. D. Osterhaus, R. de Groot, N. Corvaia, A. Beck, N. Bouveret-Le-Cam, and J. Y. Bonnefoy. 2001. Safety and immunogenicity of a novel recombinant subunit respiratory syncytial virus vaccine (BBG2Na) in healthy young adults. J. Infect. Dis. 184:1456.

45. Stott, E. J., G. Taylor, L. A. Ball, K. Anderson, K. K. Young, A. M. King, and G. W. Wertz. 1987. Immune and histopathological responses in animals vaccinated with recombinant vaccinia viruses that express individual genes of human respiratory syncytial virus. J. Virol. 61:3855.

46. Tristram, D. A., R. C. Welliver, C. K. Mohar, D. A. Hogerman, S. W. Hildreth, and P. Paradiso. 1993. Immunogenicity and safety of respiratory syncytial virus subunit vaccine in seropositive children 18-36 months old. J. Infect. Dis. 167:191.

47. Bembridge, C. P., N. Rodriguez, R. Garcia-Beato, C. Nicolson, J. A. Melero, and G. Taylor. 2000. Respiratory syncytial virus infection of gene gun vaccinated mice induces Th2-driven pulmonary eosinophilia even in the absence of sensitisation to the fusion (F) or attachment (G) protein. Vaccine 19:1038.

48. Yoshida, A., T. Nagata, M. Uchijima, T. Higashi, and Y. Koide. 2000. Advantage of gene gun-mediated over intramuscular inoculation of plasmid DNA vaccine in reproducible induction of specific immune responses. Vaccine 18:1725.

49. Livingston, J. B., S. Lu, H. Robinson, and D. J. Anderson. 1998. Immunization of the female genital tract with a DNA-based vaccine. Infect. Immun. 66:322.

50. Orson, F. M., B. M. Kinsey, P. J. Hua, B. S. Bhogal, C. L. Densmore, and M. A. Barry. 2000. Genetic immunization with lung-targeting macroaggregated polyethyleneimine-albumin conjugates elicits combined systemic and mucosal immune responses. J. Immunol. 164:6313.

51. Colombetti, L. G., S. Moerlien, and S. Pinsky. 1975. Rapid and reliable preparation of macroaggregated albumin suitable for lung scintigraphy. Int. J Nucl. Med. Biol. 2:180.

52. Hapke, E. J., and H. J. Pederson. 1969. Ultrastructural changes in rat lungs induced by radioactive macroaggregated albumin. Am. Rev. Respir. Dis. 100:194.

53. Makrigiorgos, G. M., S. J. Adelstein, and A. I. Kassis. 1990. Cellular radiation dosimetry and its implications for estimation of radiation risks. Illustrative results with technetium 99 m-labeled microspheres and macroaggregates. JAMA 264:592.

54. DeLand, F. H. 1966. The fate of macroaggregated albumin used in lung scanning. J. Nucl. Med. 7:883.

55. Knowles, M. R., K. W. Hohneker, Z. Zhou, J. C. Olsen, T. L. Noah, P. C. Hu, M. W. Leigh, J. F. Engelhardt, L. J. Edwards, K. R. Jones, and et al. 1995. A controlled study of adenoviral-vector-mediated gene transfer in the nasal epithelium of patients with cystic fibrosis. N. Engl. J. Med. 333:823.

56. Wang, H., M. N. Griffiths, D. R. Burton, and P. Ghazal. 2000. Rapid antibody responses by low-dose, single-step, dendritic cell-targeted immunization. Proc. Natl. Acad. Sci. U. S. A. 97:847.

57. Gautam, A., C. L. Densmore, B. Xu, and J. C. Waldrep. 2000. Enhanced gene expression in mouse lung after PEI-DNA aerosol delivery. Mol Ther 2:63.

58. Gautam, A., C. L. Densmore, and J. C. Waldrep. 2001. Pulmonary cytokine responses associated with PEI-DNA aerosol gene therapy. Gene Ther. 8:254.

59. Densmore, C. L., F. M. Orson, B. Xu, B. M. Kinsey, J. C. Waldrep, P. Hua, B. Bhogal, and V. Knight. 2000. Aerosol delivery of robust polyethyleneimine-DNA complexes for gene therapy and genetic immunization. Mol Ther 1:180.

60. Tripp, R. A., D. Moore, J. Winter, and L. J. Anderson. 2000. Respiratory syncytial virus infection and G and/or SH protein expression contribute to substance P, which mediates inflammation and enhanced pulmonary disease in BALB/c mice. J. Virol. 74:1614.

61. Tripp, R. A., D. Moore, L. Jones, W. Sullender, J. Winter, and L. J. Anderson. 1999. Respiratory syncytial virus G and/or SH protein alters Th1 cytokines, natural killer cells, and neutrophils responding to pulmonary infection in BALB/c mice. J Virol. 73:7099.

62. Sullender, W. 1995. Antigenic analysis of chimeric and truncated G proteins of respiratory syncytial virus. Virology 209:70.

63. Richardson, L. S., R. H. Yolken, R. B. Belshe, E. Camargo, H. W. Kim, and R. M. Chanock. 1978. Enzyme-linked immunosorbent assay for measurement of serological response to respiratory syncytial virus infection. Infect. Immun. 20:660.

64. Tripp, R. A., L. P. Jones, L. M. Haynes, H. Zheng, P. M. Murphy, and L. J. Anderson. 2001. CX3C chemokine mimicry by respiratory syncytial virus G glycoprotein. Nat Immunol 2:732.

65. Hyland, L., S. Hou, C. Coleclough, T. Takimoto, and P. C. Doherty. 1994. Mice lacking CD8+ T cells develop greater numbers of IgA-producing cells in response to a respiratory virus infection. Virology 204:234.

66. Hancock, G. E., K. M. Heers, J. D. Smith, C. A. Scheuer, A. R. lbraghimov, and K. S. Pryharski. 2001. CpG containing oligodeoxynucleotides are potent adjuvants for parenteral vaccination with the fusion (F) protein of respiratory syncytial virus (RSV). Vaccine 19:4874.

67. Simmons, C. P., T. Hussell, T. Sparer, G. Walzl, P. Openshaw, and G. Dougan. 2001. Mucosal delivery of a respiratory syncytial virus CTL peptide with enterotoxin-based adjuvants elicits protective, immunopathogenic, and immunoregulatory antiviral CD8+ T cell responses. J. Immunol. 166:1106.

68. Klinguer, C., A. Beck, P. De-Lys, M. C. Bussat, A. Blaecke, F. Derouet, J. Y. Bonnefoy, T. N. Nguyen, N. Corvaia, and D. Velin. 2001. Lipophilic quaternary ammonium salt acts as a mucosal adjuvant when co-administered by the nasal route with vaccine antigens. Vaccine 19:4236.

69. Magnusson, M., E. Johansson, M. Berg, M. L. Eloranta, L. Fuxler, and C. Fossum. 2001. The plasmid pcDNA3 differentially induces production of interferon-alpha and interleukin-6 in cultures of porcine leukocytes. Vet. Immunol. Immunopathol. 78:45.

70. McCluskie, M. J., and H. L. Davis. 2000. Oral, intrarectal and intranasal immunizations using CpG and non-CpG oligodeoxynucleotides as adjuvants. Vaccine 19:413.

71. Mitani et al. “Transduction of human bone marrow by adenoviral vector.” Human Gene Therapy 5:941-948 (1994).

72. Goodman et al. “Recombinant adeno-associated virus-mediated gene transfer into hematopoietic progenitor cells” Blood 84:1492-1500 (1994).

73. Naldini et al. “In vivo gene delivery and stable transduction of nondividing cells by a lentiviral vector” Science 272:263-267 (1996).

74. Agrawal et al. Exp. Hematol. 24:738-747 (1996).

75. Piedra P A, Camussi G, Ogra P L. Immune response to experimentally induced infection with respiratory syncytial virus: possible role in the development of pulmonary disease. J Gen Virol 1989; 70 (Pt 2):325-33.

76. Tripp R A, Anderson L J. Cytotoxic T-lymphocyte precursor frequencies in BALB/c mice after acute respiratory syncytial virus (RSV) infection or immunization with a formalin-inactivated RSV vaccine. J Virol 1998; 72(11):8971-75.

77. Magnusson M, Johansson E, Berg M, Eloranta M L, Fuxler L, Fossum C. The plasmid pcDNA3 differentially induces production of interferon-alpha and interleukin-6 in cultures of porcine leukocytes. Vet Immunol Immunopathol 2001; 78(1):45-56.

78. McCluskie M J, Davis H L. Oral, intrarectal and intranasal immunizations using CpG and non-CpG oligodeoxynucleotides as adjuvants. Vaccine 2000; 19(4-5):413-22.

79. Heeg K, Zimmermann S. CpG DNA as a Th1 trigger. Int Arch Allergy Immunol 2000; 121(2):87-97.

80. Hancock G E, Heers K M, Smith J D, Scheuer C A, lbraghimov A R, Pryharski K S. CpG containing oligodeoxynucleotides are potent adjuvants for parenteral vaccination with the fusion (F) protein of respiratory syncytial virus (RSV). Vaccine 2001; 19(32):4874-82.

TABLE 1
The percentage of cell subsets in the BAL following RSV
challenge of MAA or MAA-F vaccinated mice^{a}.

| Vaccination | Days pi | Lymphocytes^{h} | Macrophages^{b} | PMNs^{b} | Eosinophils^{b} |
|---|---|---|---|---|---|
| 1X Vaccination | | | | | |
| MAA | Pre | 18.7 ± 0.8 | 79.1 ± 4 | 2.2 ± 2.4 | 0 ± 0.9 |
| | 4 | 17.3 ± 5.2 | 74.9 ± 6 | 2.9 ± 1.2 | 3.1 ± 1.2 |
| | 14 | 15.2 ± 1.6 | 79.2 ± 5. | 4.6 ± 2 | 1 ± 0.1 |
| MAA-F | Pre | 24.8 ± 2.8^{c} | 74.3 ± 3 | 0.9 ± 0.5 | 0 |
| | 4 | 17.3 ± 2 | 82.7 ± 4 | 1.3 ± 0.7 | 1.3 ± 0.6 |
| | 14 | 15.2 ± 2.5 | 79.7 ± 2 | 0.6 ± 0 | 0 ± 0.7 |
| 2X Vaccination | | | | | |
| MAA | Pre | 38.7 ± 5 | 36.6 ± 6 | 17.3 ± 4 | 0.3 ± 0.3 |
| | 4 | 55 ± 7 | 35.2 ± 6 | 11.7 ± 2 | 0.6 ± 0.5 |
| | 12 | 56.6 ± 2 | 28.5 ± 3 | 12.3 ± 1 | 1.1 ± 0.4 |
| MAA-F | Pre | 44.2 ± 3 | 32.5 ± 7 | 22.3 ± 4 | 0.6 ± 0.5 |
| | 4 | 52.6 ± 2 | 33.8 ± 3 | 16.2 ± 2 | 0.5 ± 0.7 |
| | 12 | 56.1 ± 5 | 31.6 ± 4 | 10.5 ± 1 | 1 ± 0.3 |
| 1X Vaccination, 10 μg | | | | | |
| MAA | Pre | 40.4 ± 2 | 54.4 ± 1 | 3.1 ± 1 | 2.0 ± 0.6 |
| | 4 | 44.0 ± 11 | 48.2 ± 9 | 3.4 ± 2 | 4.4 ± 0.4 |
| | 14 | 59.7 ± 2 | 35.2 ± 3 | 2.5 ± 0.3 | 2.6 ± 1 |
| MAA-F | Pre | 33.1 ± 2 | 60.6 ± 0.5 | 2.1 ± 1 | 4.2 ± 2 |
| | 4 | 55.2 ± 0.2 | 39.3 ± 2 | 3.6 ± 3 | 2.0 ± 0.2 |
| | 14 | 68.9 ± 1.5 | 25.8 ± 1 | 3.7 ± 2 | 1.6 ± 0.8 |
| ^{a}Mice were vaccinated with MAA or MAA-F and rested 2 weeks before RSV/A2 challenge or vaccine boost. Mice that were boosted were rested 2 weeks prior to | | | | | |
| # RSV/A2 challenge. Three separate BAL samples pooled from 2 mice/treatment (6 mice for each time point) were examined for each data set. BAL cells were fixed and stained | | | | | |
| # with hematoxylin and eosin and populations of lymphocytes, macrophages, PMNs and eosinophils were scored. | | | | | |

^{b}Data are presented as average percentage of cells examined ± standard deviation.
^{c}Significant differences in the percentages of infiltrating cells were determined by unpaired, two-tail analysis (p < 0.05) comparing MAA to MAA-F vaccinated mice, and are indicated in bold text.

What is claimed is:

1. A composition comprising: 1) macroaggregated albumin, 2) a nucleic acid comprising a nucleotide sequence encoding an RSV protein and 3) polyethylamine (PEI), wherein the MAA, PEI and nucleic acid form a complex.

2. The composition of claim 1, wherein the nucleic acid is in a vector.

3. The composition of claim 1, wherein the RSV protein is an RSV/A2 F glycoprotein.

4. The composition of claim 1, wherein the RSV protein is an RSV/A2 G glycoprotein.

5. The composition of claim 1 in a pharmaceutically acceptable carrier.

6. The composition of claim 1, wherein the RSV protein is selected from the group consisting of an RSV/A1 glycoprotein, an RSV/A-Long glycoprotein, an RSV/A2 glycoprotein, an RSV/A-WV6973 glycoprotein, an RSV/B1 glycoprotein, an RSV/B-8/60 glycoprotein, an RSV/B- 18537 glycoprotein, a RSV/6340 glycoprotein, a cpts-248 RSV glycoprotein, a cpts-530 RSV glycoprotein, a Ts-1 RSV glycoprotein, and a bovine RSV/A glycoprotein.

7. A method of preventing respiratory syncytial virus (RSV) infection in a subject comprising administering to the subject an amount of the composition of claim 5 effective in preventing RSV infection in the subject.

8. The method of claim 7, wherein the subject is human.

9. The method of claim 7, wherein the composition is intranasally administered.

10. The method of claim 7, wherein the composition is intratracheally administered.

11. The method of claim 7, wherein the composition is intravenously administered.

12. A method of producing an immune response to respiratory syncytial virus (RSV) in a subject comprising administering to the subject the composition of claim 5 in an amount effective in producing an immune response to RSV in the subject.

13. The method of claim 12, wherein the subject is human.

14. The method of claim 12, wherein the composition of is intranasally administered.

15. The method of claim 12, wherein the composition of is intratracheally administered.

16. The method of claim 12, wherein the composition is intravenously administered.

17. A method of enhancing an immune response to respiratory syncytial virus (RSV) in a subject comprising administering to the subject the composition of claim 5 in an amount effective in enhancing an immune response to RSV in the subject.

18. The method of claim 17, wherein the subject is human.

19. The method of claim 17, wherein the composition is intranasally administered.

20. The method of claim 17, wherein the composition is intratracheally administered.

21. The method of claim 17, wherein the composition is intravenously administered.
